(19) United States
(12) Patent Application Publication
(10) Pub. No.: US 2005/0154356 A1
(43) Pub. Date: Jul. 14, 2005

(54) Controlled gastric bolus feeder

(76) Inventor: Lloyd Jay Pinel, Silverado, CA (US)

Correspondence Address: LLOYD J. PINEL, P.O. BOX 442, SILVERADO, CA, 92676, US

(21) Appl. No.: 10/755,992

(22) Filed: Jan. 13, 2004

(51) Int. Cl. A61M 5/00

(52) U.S. Cl. 604/247

(57) ABSTRACT

A gravity bolus feeding device for persons with gastric feeding tubes which does not require a person to manually raise and lower the fluid container to regulate the rate of flow. The feeding device can be hung from a crib, chair back or a line with the flow rate set. Venting of air and stomach contents is allowed through means of a check valve. When venting occurs fluid will not splash out of the top with burp baffle in place.

### BACKGROUND OF THE INVENTION—FIELD OF INVENTION

This invention relates to gravity bolus feeding of persons with gastric feeding tubes.

### BACKGROUND OF THE INVENTION

This invention resides in an improved device for bolus feeding persons with gastric feeding tubes. Currently there are three common methods for feeding persons with gastric feeding tubes:
1. Gravity feeding with an intravenous type bag and tubing.
2. Utilizing a feeding pump with bag and tubing.
3. Gravity bolus feeding using a syringe barrel.

For many persons with gastric feeding tubes, it is important to be able to vent air and stomach contents back up the gastric tube during and after a feeding. Methods 1 and 2 above are commonly used in the hospital setting and can benefit from the device described in U.S. Pat. No. 6,482,170. The most common method used outside of the hospital setting that allows air and stomach contents to vent is method 3 the gravity bolus method using a syringe barrel. With this method a syringe barrel is connected to the gastric feeding tube and is filled with liquid food. The syringe barrel is then raised and lowered by hand to control the rate of feeding. The open tube allows air and stomach contents to vent.

This method suffers from a number of disadvantages:
(a) Requires constant attention of the person holding the syringe during the twenty to forty minutes a typical feeding takes.
(b) Holding the syringe barrel too high results in too rapid of a feeding which can cause reflux of stomach contents and possible aspiration.
(c) Holding the syringe barrel too low causes the liquid food to return to the syringe barrel which results in an increased feeding time.
(d) Once the feeding is started it is difficult to interrupt or stop if necessary.
(e) When venting occurs the contents of the syringe barrel are often splashed out of the open top.

### BACKGROUND OF THE INVENTION—OBJECTS AND ADVANTAGES

Several objects and advantages of the present invention are:
(a) To provide a gravity bolus feeding system that does not require a person to manually raise and lower the device to control flow.
(b) To provide a gravity bolus feeding system which will permit air and stomach contents to vent during and after the feeding.
(c) To provide a gravity bolus feeding system with a top that will allow air to enter and exit without allowing fluid to splash out.
(d) To provide a gravity bolus feeding system with a means for shutoff which will allow for feeding to be stopped and disconnected as necessary.

### Description of Prior Art

A cylindrical syringe barrel attached to a gastric feeding tube is hand held and is raised or lowered to control the rate of flow of the liquid food. The tube remains open to the syringe barrel to allow for venting of air and stomach contents. Venting often causes fluid to splash out of the top of the syringe barrel.

### SUMMARY OF THE INVENTION

The present invention resides in an improved device for gravity feeding persons with gastric feeding tubes. A cylinder to be connected to a gastric feeding tube has three connecting paths for fluid which has a control to regulate the flow. The lower path will permit fluid to flow to or from the stomach while the third path with a check valve will relieve pressure from the lower path when venting occurs. An upper burp baffle with path having 90 degree bends will allow air to enter and exit but prevent fluid from splashing out.

### BREIF DESCRIPTION OF DRAWINGS

FIG. 1 Is a cross-sectional drawing of a syringe barrel.

FIG. 2 Is a cross-sectional drawing of a burp baffle.

FIG. 3 Is an end view of top section of FIG. 2.

FIG. 4 Is an end view of the middle section of FIG. 2.

FIG. 5 Is an end view of bottom section of FIG. 2.

FIG. 6 Is a cross-sectional assembly drawing of the invention.

### DETAILED DESCRIPTION OF THE INVENTION

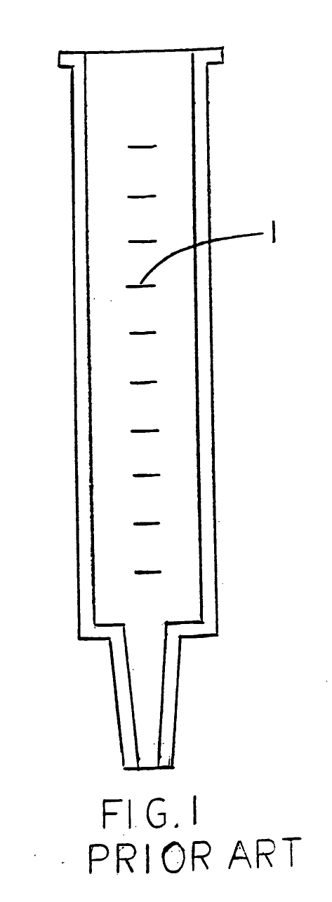

FIG. 1 Is a cross-sectional drawing of a syringe barrel with quantity markings.

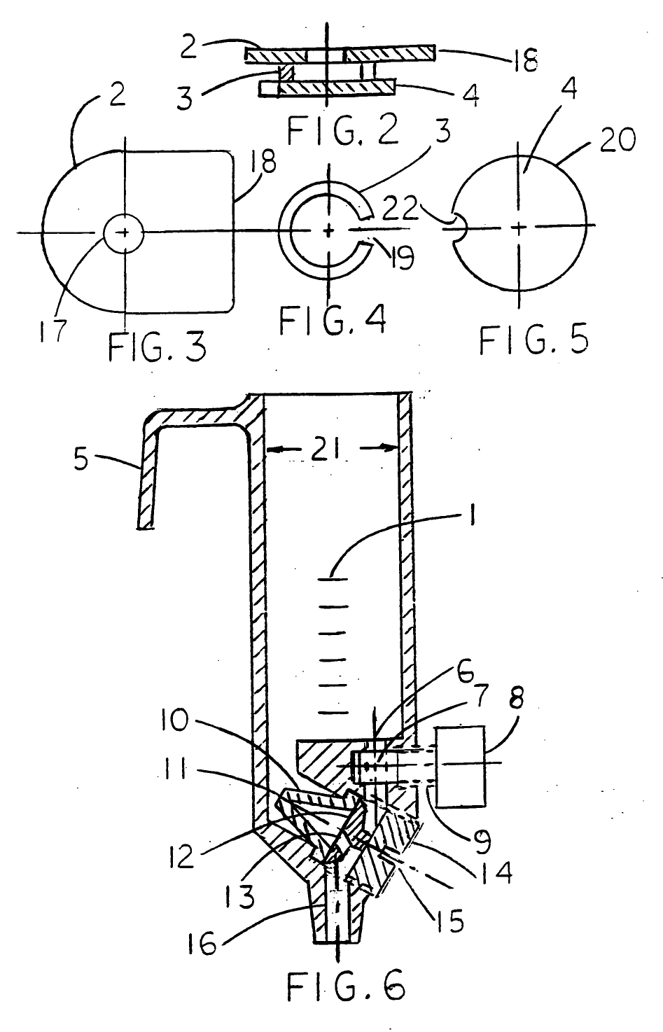

FIG. 2 Is a cross-sectional drawing of a burp baffle with top section 2 having hole 17 for air intake and vent and flat 18 to prevent spillage when laid down.

FIG. 3 Is an end view of top section 2 with hole 17 and flat 18.

FIG. 4 Is an end view of middle section 3 shaped to baffle with opening 19 for air vent and intake. The three sections create a flow path with 90 degree bends.

FIG. 5 Is an end view of bottom section 4 with outer diameter 20 to fit into inner diameter 21 with small arc 22 for air vent and intake.

FIG. 6 Is a cross-sectional drawing of a gravity bolus feeding device with an inner diameter 21 to hold the liquid food with quantity markings 1. Hook handle 5 will allow feeder to hang from crib rail, chair back or line. Liquid food may flow through hole 6 and hole 7 controlled by twist of knob 8 with threads 9. Flow will continue by check valve spacer 12 and out through hole 16. Check valve spacer 12 with back flow holes 13 is held in place by set screw 14 having slot 15 for tightening. Rubber check valve 10 opens at slot 11 with back pressure to permit flow which will not splash out with burp baffle in place.

### Operation FIGS. 2 and 6

Begin use by connecting said feeding device FIG. 6 to a clamped and primed feeding tube or extension tube approximately 12 inches long. With control knob 8 in the closed position, fill inner diameter 21 with liquid food to the desired quantity marking 1. Insert burp baffle FIG. 2 into inner diameter 21 with flat 18 positioned opposite of handle 5. Hang said feeding device from crib rail, chair back or line approximately 12 to 18 inches above stomach level. Open feeding tube clamp. Open control knob 8 to set desired feeding rate. If venting occurs from excess gastric pressure, air and stomach contents will return to inner diameter 21 through check valve spacer back flow holes 13 and check valve slot 11. Air which returns to inner diameter 21 will separate out and be exhausted through burp baffle FIG. 2. If it becomes necessary to stop feeding and disconnect the feeding device, turn control knob 8 to off. Clamp feeding tube and disconnect the feeding device. The feeding device may be laid down on flat 18 and fluid will not spill out.

1. A gravity bolus feeding device which comprises three joining fluid paths where the intake is controllable for the quantity of flow in while one path the flow can be in or out and the third path has a check valve to relieve the pressure from the in or out path.

2. A burp baffle to prevent splash out with a path for intake and vent having 90 degree bends.
